(19) United States
(12) Patent Application Publication
(10) Pub. No.: US 2025/0345560 A1
(43) Pub. Date: Nov. 13, 2025

(54) VENOUS CATHETER

(71) Applicant: TERUMO KABUSHIKI KAISHA, Tokyo (JP)

(72) Inventors: Takashi ITO, Shizuoka-shi (JP); Mitsuteru YASUNAGA, Fujinomiya-shi (JP); Toshiki ASAWA, Fujinomiya-shi (JP)

(73) Assignee: TERUMO KABUSHIKI KAISHA, Tokyo (JP)

(21) Appl. No.: 19/273,850

(22) Filed: Jul. 18, 2025

Related U.S. Application Data
(63) Continuation of application No. PCT/JP2023/041991, filed on Nov. 22, 2023.

(30) Foreign Application Priority Data
Jan. 20, 2023 (JP) 2023-007032

(51) Int. Cl. A61M 25/00; A61M 25/09

(52) U.S. Cl. CPC A61M 25/005; A61M 25/0097; A61M 25/09; A61M 2025/0059

(57) ABSTRACT

To provide a venous catheter that is easily inserted into a vein and has favorable lesion passability. A venous catheter has a shaft portion having a long tubular shape, and is inserted into a vein of a living body. In the venous catheter, the shaft portion has: a base portion having a constant outer diameter along a long axis direction; and a tapered portion having a tapered outer diameter from a distal end of the base portion to a distal end of the shaft portion, an inner diameter of the base portion is 0.86 mm or more and 1.2 mm or less, an outer diameter of the base portion is 1.5 mm or more and 2.2 mm or less, and a length of the tapered portion along a long axis direction is 10 mm or more.

### CROSS-REFERENCES TO RELATED APPLICATIONS

This application is a continuation of International Application No. PCT/JP2023/041991 filed on Nov. 22, 2023, which claims priority to Japanese Patent Application No. 2023-007032 filed on Jan. 20, 2023, the entire content of both of which is incorporated herein by reference.

### TECHNOLOGICAL FIELD

The present invention generally relates to a venous catheter that is used by being inserted into a vein of a living body.

### BACKGROUND DISCUSSION

As a method of treating a lesion such as stenosis or occlusion generated in a blood vessel, there is endovascular treatment in which treatment is performed from inside the blood vessel using a device percutaneously inserted into the blood vessel. In the endovascular treatment, a catheter is used to cause a contrast medium for diagnosis, a guide wire, or the like to reach the lesion.

International Patent Application Publication No. 2018/092387 (WO 2018/092387 A) discloses a catheter that is inserted into a lumen of a guiding catheter to improve kink resistance and reachability of the guiding catheter. The catheter has a tapered portion having a tapered shape at a distal portion.

### SUMMARY

In treatment of deep vein thrombosis, a guide wire is configured to pass through a thrombus generated in an iliac vein, a femoral vein, and a popliteal vein. A support catheter is used to support passage of the guide wire through a lesion in this case. Conventionally, the support catheter for an artery is known. For this reason, the support catheter for an artery has been used also in treatment of veins.

A vein has a larger vessel diameter than an artery. Therefore, when the support catheter for an artery is inserted into a vein, a large gap is generated between an outer peripheral surface of the support catheter and an inner wall of the vein, so that the support catheter is easily deflected in a vein. For this reason, a device specialized for passing through a lesion in a vein is required.

The venous catheter disclosed here is easily inserted into a vein and also has favorable lesion passability.

(1) The venous catheter that can be easily inserted into a vein and that exhibits favorable lesion passability has a shaft portion having a long tubular shape and is inserted into a vein of a living body, in which the shaft portion has: a base portion having a constant outer diameter along a long axis direction; and a tapered portion having a tapered outer diameter from a distal end of the base portion to a distal end of the shaft portion, an inner diameter of the base portion is 0.86 mm or more and 1.2 mm or less, an outer diameter of the base portion is 1.5 mm or more and 2.2 mm or less, and a length of the tapered portion along the long axis (longitudinal axis) direction is 10 mm or more.

Since the venous catheter configured as described above has an outer diameter suitable for an inner diameter of a vein into which the venous catheter is inserted, it is possible to reduce a gap between an outer peripheral surface of the shaft portion and an inner wall of the vein at the time of insertion. In addition, since a wall thickness of the shaft portion at the base portion is large, rigidity is high, and deflection in a vein can be suppressed, so that insertion can be facilitated.

(2) In the venous catheter of (1) above, an outer diameter of the distal end of the shaft portion may be 1.05 mm, and a length of the tapered portion along the long axis (longitudinal axis) direction may be 10 mm or more and 20 mm or less. As a result, the tapered portion can be formed at a long and gentle inclination angle along the long axis direction, and passability in a lesion can be improved.

(3) In the venous catheter of (1) above, a length of the tapered portion of the shaft portion along the long axis direction may be more than 12 mm. As a result, a length of the tapered portion along the long axis direction can be further increased, and passability in a lesion can be further improved.

According to another aspect, a venous catheter comprises: a shaft portion having a proximal end and a distal end, a hub connected to a proximal end of the shaft portion, and a kink protector surrounding a portion of the shaft portion and a portion of the hub. The shaft portion is configured to be inserted into a vein of a living body, the shaft portion has a tubular shape along its entire extent and is elongated along a longitudinal axis direction of the shaft portion. The shaft portion includes a base portion and a tapered portion, with the base portion having proximal and distal ends, and the base portion having a constant outer diameter along the longitudinal axis direction of the shaft portion from the proximal end of the shaft portion to the distal end of the shaft portion, and the tapered portion having proximal and distal ends. The proximal end of the tapered portion is connected to the distal end of the base portion, the tapered portion has a tapered outer diameter that tapers in a narrowing manner from the proximal end of the tapered portion to the distal end of the tapered portion which is the distal end of the shaft portion. The inner diameter of the base portion is 0.86 mm or more and 1.2 mm or less, the outer diameter of the base portion is 1.5 mm or more and 2.2 mm or less, and the length of the tapered portion along the longitudinal axis direction is 10 mm or more. The shaft portion includes a reinforcing body positioned between inner and outer surfaces of the shaft portion, with the reinforcing body extending along the longitudinal axis direction of the shaft portion, and the shaft portion has a length, from a most distal end of the kink protector to a most distal end of the shaft portion, that is 600 mm to 1350 mm.

According to another aspect, a method comprises inserting a venous catheter into a vein of a living body for subsequent passage through a lesion. The venous catheter comprises: a shaft portion configured to be inserted into a vein of a living body, with the shaft portion having an elongated tubular shape that is elongated along a longitudinal axis direction of the shaft portion, the shaft portion having a length extending from a distal end of the shaft portion to a proximal end of the shaft portion, and the shaft portion including a lumen that extends throughout the shaft. The shaft portion includes both a base portion and a tapered portion, with the base portion having a constant outer diameter along the longitudinal axis direction, and the tapered portion having a tapered outer diameter from a distal end of the base portion to a distal end of the shaft portion. The base portion has an inner diameter that is 0.86 mm or more and 1.2 mm or less, the base portion has an outer diameter that is 1.5 mm or more and 2.2 mm or less, and the tapered portion has a length along the longitudinal axis direction that is 10 mm or more. The method additionally involves inserting a guide wire into the lumen in the shaft portion when the venous catheter is positioned in the vein of the living body.

### BRIEF DESCRIPTION OF THE DRAWINGS

FIG. 1 is an overall view of a catheter according to the present embodiment.

FIG. 2 is an enlarged crosssectional view illustrating a portion of a proximal shaft of a shaft portion of the catheter.

FIG. 3 is an enlarged front view illustrating a part of a reinforcing layer.

FIG. 4 is an enlarged view illustrating a distal portion of the shaft portion of the catheter in an enlarged manner.

### DETAILED DESCRIPTION

An embodiment of the venous catheter disclosed here, representing an example of the new venous catheter, will be described hereinafter with reference to the drawings. dimensions in the drawings may be exaggerated and different from actual dimensions for convenience of description and ease of understanding. In the present specification and the drawings, components having substantially the same functional configuration are denoted by the same reference numerals, and a detailed description of already described features will not be repeated. In the present specification, in a venous catheter, a side to be inserted into a blood vessel is referred to as a “distal side”, and a side to be operated is referred to as a “proximal side”.

In the description of the present specification, a direction in which the venous catheter extends in a natural state (straight state with no external force applied) is referred to as a “long axis direction” or a “longitudinal axis direction”. A rotation direction with the long axis direction of the venous catheter as a reference axis is referred to as a “circumferential direction”. In addition, in the venous catheter, a side to be inserted into a blood vessel is defined as a distal side, and an end side opposite to the distal side is defined as a proximal side. A portion including a certain range in the long axis (longitudinal axis) direction from a distal end (most distal end) is referred to as a “distal portion”, and a portion including a certain range in the long axis (longitudinal axis) direction from a proximal end (most proximal end) is referred to as a “proximal portion”. Here, left and right rotation directions are defined such that a clockwise direction is the right rotation direction and a counterclockwise direction is the left rotation direction, as viewed from the proximal side with the long axis as a center.

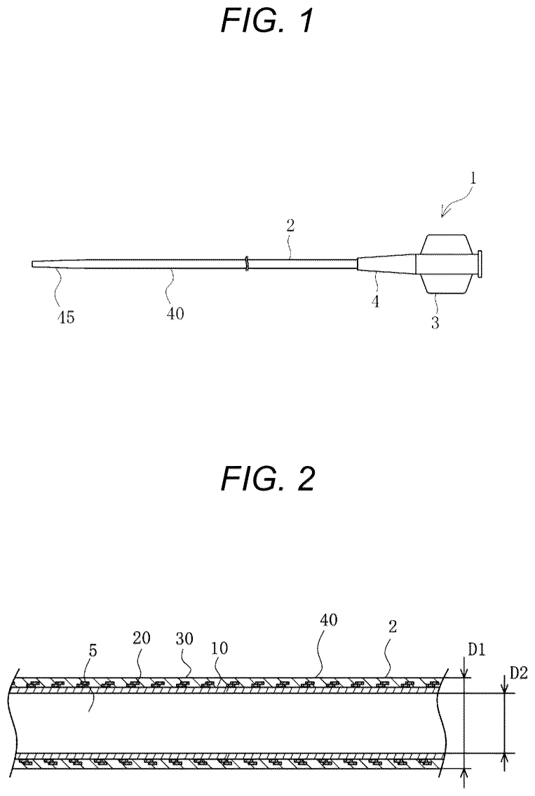

A venous catheter 1 according to the present embodiment is insertable into any of an iliac vein, a femoral vein, and a popliteal vein, and is used to, and specifically configured to, support passage of a guide wire through a lesion. As illustrated in FIG. 1, the venous catheter 1 has a shaft portion 2 having a long tubular shape (elongated tubular shape), a hub 3 connected to a proximal end of the shaft portion 2, and a kink protector 4 surrounding a connection portion between the shaft portion 2 and the hub 3.

The shaft portion 2 is a tubular member having flexibility, and a lumen 5 is formed inside the shaft portion 2, extending from the proximal end of the shaft portion to a distal end of the shaft portion. The guide wire is inserted into the lumen 5 when the venous catheter 1 is inserted into a blood vessel.

An effective length of the shaft portion 2 is not particularly limited, but is 600 mm to 2500 mm, for example. The effective length of the shaft portion 2 is a length of a portion that can be inserted into a blood vessel or a sheath, and is more preferably 600 mm to 1350 mm. In the present embodiment, the effective length is a length from the most distal end of the kink protector 4 to the most distal end of the shaft portion 2, and is 900 mm.

The shaft portion 2 has a base portion 40 having a constant outer diameter along the long axis (longitudinal axis) direction, and a tapered portion 45 having a tapered outer diameter that tapers in a narrowing manner from a distal end of the base portion 40 to the distal end of the shaft portion 2. The base portion 40 extends from the kink protector 4 to the distal side (in the distal direction), and occupies most of the length of the shaft portion 2 in the long axis (longitudinal axis) direction.

The hub 3 is liquid-tightly fixed to the proximal portion of the shaft portion 2 with an adhesive, heat fusion, a stopper (not illustrated), or the like. The hub 3 functions as an insertion port for the guide wire into the lumen 5. The hub 3 also functions as a grip portion when the venous catheter 1 is operated.

The hub 3 is not particularly limited, but may be formed of a resin such as polycarbonate, polyamide, polysulfone, polyarylate, or a methacrylate-butylene-styrene copolymer.

The kink protector 4 is provided so as to surround the connection portion between the shaft portion 2 and the hub 3, and suppresses kinking of the shaft portion 2 at the connection portion between the shaft portion 2 and the hub 3. The kink protector 4 is made of an elastic material such as elastomer, natural rubber, or silicone resin.

As illustrated in FIG. 2, the shaft portion 2 includes an inner layer 10 having the lumen 5, a reinforcing body 20 disposed outside the inner layer 10, and an outer layer 30 disposed outside the inner layer 10 and the reinforcing body 20.

The inner layer 10 has the lumen 5 formed therein. The inner layer 10 is formed of a fluorine-based resin such as polytetrafluoroethylene (PTFE), or a low-friction material such as high-density polyethylene (HDPE). Alternatively, the inner layer 10 may be formed of a polymer material such as polyolefin (for example, polyethylene, polypropylene, polybutene, an ethylene-propylene copolymer, an ethylene-vinyl acetate copolymer, an ionomer, or a mixture of two or more types thereof), polyvinyl chloride, polyamide, a polyester elastomer, a polyamide elastomer, polyurethane, a polyurethane elastomer, polyimide, or a fluororesin, a thermoplastic resin such as a mixture of these, or a thermosetting resin such as an epoxy resin.

The outer layer 30 is a tubular member that covers an outer periphery of the inner layer 10 and the reinforcing body 20. The outer layer 30 is formed of a polymer material such as polyolefin (for example, polyethylene, polypropylene, polybutene, an ethylene-propylene copolymer, an ethylene-vinyl acetate copolymer, an ionomer, or a mixture of two or more types thereof), polyvinyl chloride, polyamide, a polyester elastomer, a polyamide elastomer, polyurethane, a polyurethane elastomer, polyimide, or a fluororesin, a thermoplastic resin such as a mixture of these, or a thermosetting resin such as an epoxy resin.

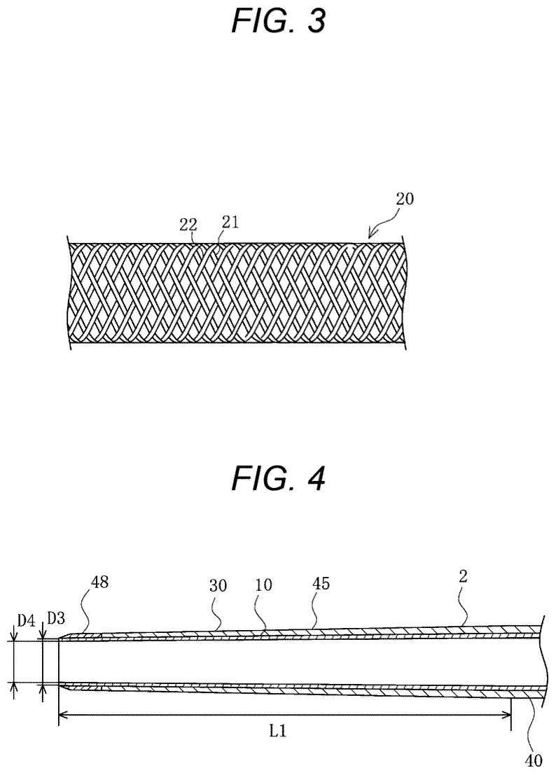

As illustrated in FIG. 3, the reinforcing body 20 is formed by knitting a first reinforcing body 21 wound in one direction of the right rotation direction or the left rotation direction with respect to the shaft portion 2, and a second reinforcing body 22 wound in another direction of the right rotation direction or the left rotation direction with respect to the shaft portion 2, that is, in an opposite direction, in a mesh shape so as to cross each other. The first reinforcing body 21 and the second reinforcing body 22 can be formed of stainless steel such as SUS304 or SUS316, tungsten, or the like. Wire diameters of the first reinforcing body 21 and the second reinforcing body 22 are 0.06 mm or more and 0.1 mm or less. In the base portion 40 of the shaft portion 2, the reinforcing body 20 is provided from the proximal end to a position on the proximal side with respect to the tapered portion 45.

As illustrated in FIG. 4, the tapered portion 45 is not provided with the reinforcing body 20. Both an outer diameter and an inner diameter of the tapered portion 45 decrease toward the distal side. A distal tip 48 is provided at a distal portion of the tapered portion 45.

Dimensions of the shaft portion 2 will be described. A vein of a lower limb into which the shaft portion 2 is inserted has an inner diameter of approximately 2 to 16 mm. In addition, a vein has characteristics of having a blood vessel wall thinner than that of an artery, being easily torn, and having many microchannels on a surface. The shaft portion 2 is set to have the following dimensions in order to be less likely to be deflected when inserted into a vein and to improve passability in a lesion. In the shaft portion 2, an outer diameter D1 of the base portion 40 is 1.5 mm or more and 2.2 mm or less, and an inner diameter D2 of the base portion 40 is 0.86 mm or more and 1.2 mm or less. An overall length (L1) of the tapered portion 45 along the long axis (longitudinal axis) direction is 10 mm or more and 20 mm or less. An outer diameter D3 at the distal end of the shaft portion 2 is 1.05 mm, and an inner diameter D4 at the distal end of the shaft portion 2 is 0.90 mm or more and 0.92 mm or less.

The shaft portion 2 of the venous catheter 1 that meets these dimensional conditions will be described in Example 1 to 4. In addition, as a comparative example, dimensions of a shaft portion of an arterial catheter are also shown.

### EXAMPLE 1

Base outer diameter (D1): 1.6 mm

Base inner diameter (D2): 1.05 mm

Distal end outer diameter (D3): 1.05 mm

Distal end inner diameter (D4): 0.90 mm

Tapered portion overall length (L1): 10 mm

### EXAMPLE 2

Base outer diameter (D1): 1.6 mm

Base inner diameter (D2): 1.05 mm

Distal end outer diameter (D3): 1.05 mm

Distal end inner diameter (D4): 0.90 mm

Tapered portion overall length (L1): 20 mm

### EXAMPLE 3

Base outer diameter (D1): 1.8 mm

Base inner diameter (D2): 1.05 mm

Distal end outer diameter (D3): 1.05 mm

Distal end inner diameter (D4): 0.90 mm

Tapered portion overall length (L1): 10 mm

### EXAMPLE 4

Base outer diameter (D1): 1.8 mm

Base inner diameter (D2): 1.05 mm

Distal end outer diameter (D3): 1.05 mm

Distal end inner diameter (D4): 0.90 mm

Tapered portion overall length (L1): 20 mm

### Comparative Example

Base outer diameter (D1): 1.4 mm

Base inner diameter (D2): 1.05 mm

Distal end outer diameter (D3): 1.05 mm

Distal end inner diameter (D4): 0.90 mm

Tapered portion overall length (L1): 12 mm

The shaft portion 2 of the venous catheter 1 of the present embodiment has the same base inner diameter D2 as the shaft portion of the arterial catheter of the comparative example, but has the large base outer diameter D1, and thus has a large wall thickness. For this reason, in the shaft portion 2 of the venous catheter 1, a gap between with the inner wall surface of the blood vessel is small at the time of insertion into the vein, and rigidity is high since the wall thickness is large. As a result, the shaft portion 2 of the venous catheter 1 is less likely to be deflected in a vein, and insertion can be facilitated. In addition, the length of the shaft portion 2 of the venous catheter 1 along the long axis (longitudinal axis) direction of the tapered portion 45 can be set to a length more than 12 mm of the arterial catheter. As a result, it is possible to further increase lesion passability in a blood vessel (i.e., the ability of the venous catheter to be passed through a lesion in a blood vessel is improved).

As described above, (1) the venous catheter 1 according to the present embodiment is the venous catheter 1 that has the shaft portion 2 having a long tubular shape (elongated tubular shape), and is inserted into a vein of a living body, in which the shaft portion 2 has: the base portion 40 having a constant outer diameter along a long axis (longitudinal axis) direction; and the tapered portion 45 having a tapered outer diameter from a distal end of the base portion 40 to a distal end of the shaft portion 2, an inner diameter of the base portion 40 is 0.86 mm or more and 1.2 mm or less, an outer diameter of the base portion 40 is 1.5 mm or more and 2.2 mm or less, and a length of the tapered portion 45 along the long axis (longitudinal axis) direction is 10 mm or more. Since the venous catheter 1 configured in this way has an outer diameter suitable for an inner diameter of a vein into which the venous catheter 1 is inserted, it is possible to reduce a gap between an outer peripheral surface of the shaft portion 2 and an inner wall of the vein at the time of insertion. In addition, since a wall thickness of the shaft portion 2 at the base portion 40 is relatively large, rigidity is high, and deflection in a vein can be suppressed, so that insertion can be facilitated.

(2) In the venous catheter 1 of (1) above, an outer diameter of the distal end of the shaft portion 2 may be 1.05 mm, and a length of the tapered portion 45 along the long axis (longitudinal axis) direction may be 10 mm or more and 20 mm or less. As a result, the tapered portion 45 can be formed at a long and gentle inclination angle along the long axis direction, and passability in a lesion can be improved.

(3) In the venous catheter 1 of (1) above, a length of the shaft portion 2 along the long axis direction of the tapered portion 45 may be more than 12 mm. As a result, the length of the tapered portion 45 along the long axis direction can be further increased, and passability in a lesion can be further improved.

The present invention is not limited to the embodiment described above, and those skilled in the art can make various modifications within the technical idea of the present invention.

### REFERENCE SIGNS LIST

1 Catheter
2 Shaft portion
3 Hub
4 Kink protector
5 Lumen
10 Inner layer
20 Reinforcing body
21 First reinforcing body
22 Second reinforcing body
30 Outer layer
40 Base portion
45 Tapered portion
48 Distal tip

What is claimed is:

1. A venous catheter comprising:
a shaft portion that is configured to be inserted into a vein of a living body, the shaft portion having an elongated tubular shape that is elongated along a longitudinal axis direction of the shaft portion,
the shaft portion including: a base portion having a constant outer diameter along the longitudinal axis direction; and a tapered portion having a tapered outer diameter from a distal end of the base portion to a distal end of the shaft portion,
an inner diameter of the base portion is 0.86 mm or more and 1.2 mm or less,
an outer diameter of the base portion is 1.5 mm or more and 2.2 mm or less, and
a length of the tapered portion along the longitudinal axis direction is 10 mm or more.

2. The venous catheter according to claim 1, wherein an outer diameter of a distal end of the shaft portion is 1.05 mm, and a length of the tapered portion along a long axis direction is 10 mm or more and 20 mm or less.

3. The venous catheter according to claim 1, wherein a length of the tapered portion of the shaft portion along a long axis direction is more than 12 mm.

4. The venous catheter according to claim 1, wherein the shaft portion includes a proximal end opposite the distal end of the shaft portion, and further comprising a hub connected to the proximal end of the shaft portion.

5. The venous catheter according to claim 4, wherein the hub is connected to the proximal end of the shaft portion at a connection portion, further comprising a kink protector surrounding the connection portion to suppress kinking of the shaft portion at the connection portion.

6. A venous catheter comprising:
a shaft portion having a proximal end and a distal end,
a hub connected to a proximal end of the shaft portion,
a kink protector surrounding a portion of the shaft portion and a portion of the hub,
the shaft portion being configured to be inserted into a vein of a living body, the shaft portion having a tubular shape along its entire extent and being elongated along a longitudinal axis direction of the shaft portion,
the shaft portion including a base portion and a tapered portion, the base portion having proximal and distal ends, the base portion having a constant outer diameter along the longitudinal axis direction of the shaft portion from the proximal end of the shaft portion to the distal end of the shaft portion, the tapered portion having proximal and distal ends, the proximal end of the tapered portion being connected to the distal end of the base portion, the tapered portion having a tapered outer diameter that tapers in a narrowing manner from the proximal end of the tapered portion to the distal end of the tapered portion which is the distal end of the shaft portion,
an inner diameter of the base portion is 0.86 mm or more and 1.2 mm or less,
an outer diameter of the base portion is 1.5 mm or more and 2.2 mm or less,
a length of the tapered portion along the longitudinal axis direction is 10 mm or more,
the shaft portion including a reinforcing body positioned between inner and outer surfaces of the shaft portion, the reinforcing body extending along the longitudinal axis direction of the shaft portion, and
the shaft portion having a length, from a most distal end of the kink protector to a most distal end of the shaft portion, that is 600 mm to 1350 mm.

7. The venous catheter according to claim 6, wherein an entirety of the tapered portion of the shaft portion is devoid of the reinforcing body.

8. The venous catheter according to claim 6, wherein the reinforcing body is comprised of first and second reinforcing bodies that are wound.

9. The venous catheter according to claim 8, wherein an entirety of the tapered portion of the shaft portion is devoid of the first and second reinforcing bodies.

10. The venous catheter according to claim 6, wherein the reinforcing body is comprised of first and second reinforcing bodies that are wound in opposite directions in a mesh shape.

11. The venous catheter according to claim 10, wherein an entirety of the tapered portion of the shaft portion is devoid of the first and second reinforcing bodies.

12. The venous catheter according to claim 6, wherein both an outer diameter and an inner diameter of the tapered portion decrease toward the distal end of the tapered portion.

13. The venous catheter according to claim 6, wherein the length of the tapered portion along the longitudinal axis direction is 10 mm or more and 20 mm or less.

14. The venous catheter according to claim 13, wherein an outer diameter of a distal end of the shaft portion is 1.05 mm.

15. The venous catheter according to claim 6, wherein an outer diameter of a distal end of the shaft portion is 1.05 mm.

16. The venous catheter according to claim 6, wherein the length of the tapered portion of the shaft portion along the longitudinal axis direction is more than 12 mm.

17. A method comprising:
inserting a venous catheter into a vein of a living body for subsequent passage through a lesion, the venous catheter comprising:
a shaft portion configured to be inserted into a vein of a living body, the shaft portion having an elongated tubular shape that is elongated along a longitudinal axis direction of the shaft portion, the shaft portion having a length extending from a distal end of the shaft portion to a proximal end of the shaft portion, the shaft portion including a lumen that extends throughout the shaft;
the shaft portion including both a base portion and a tapered portion, the base portion having a constant outer diameter along the longitudinal axis direction, the tapered portion having a tapered outer diameter from a distal end of the base portion to a distal end of the shaft portion,
the base portion having an inner diameter that is 0.86 mm or more and 1.2 mm or less,
the base portion having an outer diameter that is 1.5 mm or more and 2.2 mm or less, and
the tapered portion having a length along the longitudinal axis direction that is 10 mm or more;
inserting a guide wire into the lumen in the shaft portion when the venous catheter is positioned in the vein of the living body.

18. The method according to claim 17, wherein the vein is an iliac vein, a femoral vein or a popliteal vein.
